(19) United States
(12) Patent Application Publication
(10) Pub. No.: US 2010/0062087 A1
(43) Pub. Date: Mar. 11, 2010

(54) Nutraceutical Treatments for Diabetic and Non-Diabetic Wound Healing

(75) Inventors: Xiaoming Xu Chien, Benicia, CA (US); Debasis Bagchi, Benicia, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Interhealth Nutraceuticals, Inc., Benicia (CA)

(21) Appl. No.: 12/303,033

(22) PCT Filed: Jun. 4, 2007
(86) PCT No.: PCT/US07/70368

§ 371 Date: Oct. 14, 2009

Related U.S. Application Data
(60) Provisional application No. 60/810,741, filed on Jun. 2, 2006.

(30) Foreign Application Priority Data
May 31, 2007 (US) 11809149

(51) Int. Cl. A61K 36/45; A61K 36/185; A61K 36/886; A61K 33/30; A61K 33/24; A61K 31/555; A61K 31/375; A61K 31/047; A61K 31/198; A61K 36/00; A61P 3/10; A61P 25/00

(52) U.S. Cl. 424/732; 424/725; 424/744; 424/641; 424/655; 514/186; 514/474; 514/729; 514/565; 424/777

(57) ABSTRACT

In an embodiment of the present invention, a nutraceutical formula comprising a combination of two or more substances from the group: berry extract, chromium, zinc, trans-resveratrol, l-arginine, chlorophyll, vitamin C and aloe vera combined synergistically to reduce inflammation in mammals suffering from diabetes. In another embodiment of the present invention, a nutraceutical formula comprising a combination of two or more substances from the group: berry extract, chromium, zinc, fr-απs-resveratrol, l-arginine, chlorophyll, vitamin C and aloe vera combined synergistically to enhance wound healing in mammals suffering from diabetes. In an alternative embodiment of the present invention, a nutraceutical formula comprising a combination of two or more substances from the group: berry extract, chromium, zinc, trans-resveratrol, l-arginine, chlorophyll, vitamin C and aloe vera combined synergistically to reduce inflammation in mammals not suffering from diabetes. In another embodiment of the present invention, a nutraceutical formula comprising a combination of two or more substances from the group: berry extract, chromium, zinc, trans-resveratrol, l-arginine, chlorophyll, vitamin C and aloe vera, combined synergistically to enhance wound healing in mammals not suffering from diabetes.

### FIELD OF THE INVENTION

The invention relates to proposed nutraceutical treatments for aiding in wound healing in diabetic and non-diabetic patients.

### BACKGROUND OF THE INVENTION

Skin is the largest organ of the human body, weighing approximately 10 pounds and covering an area of about 16 square feet. Skin is responsible for protecting the internal organs from the external world. Skin protects the body from heat, cold and physical injuries. It also provides sensory information about the nature of the external world and is the first defense against invasion by bacteria, viruses and other noxious compounds. The skin is also an excretory organ, disposing of wastes from the body in order to maintain homeostasis.

The epidermis is a stratified squamous epithelium forming the barrier that excludes harmful microbes and retains body fluids. To perform these functions, proliferative basal cells in the innermost layer periodically detach from an underlying basement membrane of extracellular matrix, move outward and eventually die. Once suprabasal, cells stop dividing and enter a differentiation program to form the barrier. The mechanism of stratification is poorly understood. Although studies in vitro have led to the view that stratification occurs through the delamination and subsequent movement of epidermal cells, most culture conditions favor keratinocytes that lack the polarity and cuboidal morphology of basal keratinocytes in tissue. These features could be important in considering an alternative mechanism that stratification occurs through asymmetric cell divisions in which the mitotic spindle orients perpendicularly to the basement membrane.

The primary function of normal intact skin is to control microbial populations that reside on the skin surface and to prevent underlying tissue from becoming invaded by pathogens (Bowler, P. G., et al., “Wound Microbiology and Associated Approaches to Wound Management,” Clinical Microbiology Reviews, 244-269, April 2001, which is herein expressly incorporated by reference in its entirety). Since wound colonization is often microbial involving pathogenic microorganisms, a wound left untreated can become infected. Wounds can be categorized as either acute or chronic. Acute wounds are caused by external damage to intact skin and include surgical wounds, bites, minor cuts and abrasions, and more severe traumatic wounds such as lacerations and gun shot wounds. Chronic wounds are generally caused by endogenous conditions, such as diabetes, due to a predisposition that compromises the integrity of the dermal and epidermal tissue, immune system or other wound healing mechanisms.

### Oxygen's Role in Wound Healing

Reports from Jacques Cousteau's divers that they healed wounds significantly better when they lived in an undersea habitat about 35 feet under the surface of the Red Sea stimulated interest in the role of oxygen in wound healing (Hunt, T. K., et al., “Oxygen: at the foundation of wound healing-introduction,” World Journal of Surgery, 28:291-293, 2004 which is herein expressly incorporated by reference in its entirety). Further research led to the consensus that limited supply of oxygen to the wound site represents a key factor for healing. Recent research substantiates that, in biological tissues, oxygen generates reactive derivatives commonly referred to as Reactive Oxygen Species (ROS).

Disrupted vasculature limits the supply of oxygen to the wound-site. Compromised tissue oxygenation or wound hypoxia is viewed as a major factor that limits the healing process as well as wound disinfection. However, oxygen also fuels tissue regeneration, as well as the oxygen-dependent respiratory burst, which is a primary mechanism to resist infection.

Wound healing commences with blood coagulation followed by infiltration of neutrophils and macrophages at the wound site to destroy pathogenic organisms through the release of ROS by an oxygen-consuming respiratory burst. The wound-site has two clear sources of ROS: (i) transient delivery of larger amounts by respiratory burst of phagocytic cells; and (ii) sustained delivery of lower amounts by enzymes present in cells such as the fibroblasts, keratinocytes and endothelial cells. At low concentrations, ROS may serve as signaling messengers in the cell and regulate numerous signal transduction and gene expression processes. Inducible ROS generated in some non-phagocytic cells are implicated in mitogenic signaling.

### Circulation and Wound Healing

A wound demands a high priority of circulating substrates to fight infection and heal the wound. Adequate circulation is required to transport antibodies, enzymes, neutrophils, phagocytes and other white blood cells at the wound site to destroy pathogenic organisms and fight infection. In the case of tissue repair, intricate shuttling mechanisms are required to transport substrates such as amino acids, vitamins and minerals required for protein and collagen synthesis at the wound site. It also requires an exit route for excretion of metabolites and toxins resulting from infection and wound healing processes. Poor peripheral circulation due to hyperlipdemia, plaque build-up or neuropathy can impede circulation and obstruct the body's ability to fight infection and heal wounds.

### Diabetes Mellitus

Diabetes mellitus is a group of diseases characterized by high levels of blood glucose resulting from defects in insulin production, insulin action, or both. Diabetes can be associated with serious complications and premature death, but people with diabetes can take steps to control the disease and lower the risk of complications.

There are three main types of diabetes: Type I, Type II and Gestational diabetes. Type I diabetes was previously called insulin-dependent diabetes mellitus (IDDM) or juvenile-onset diabetes. Type I diabetes develops when the body's immune system destroys pancreatic beta cells, the only cells in the body that make the hormone insulin that regulates blood glucose. This form of diabetes usually strikes children and young adults, although disease onset can occur at any age. Type I diabetes may account for 5 percent to 10 percent of all diagnosed cases of diabetes. Risk factors for type I diabetes may include autoimmune, genetic and environmental factors.

Type II diabetes was previously called non-insulin-dependent diabetes mellitus (NIDDM) or adult-onset diabetes. Type II diabetes may account for about 90 percent to 95 percent of all diagnosed cases of diabetes. It usually begins as insulin resistance, a disorder in which the cells do not use insulin properly. As the need for insulin rises, the pancreas gradually loses its ability to produce insulin. Type II diabetes is associated with older age, obesity, family history of diabetes, history of gestational diabetes, impaired glucose metabolism, physical inactivity, and race/ethnicity. African Americans, Hispanic/Latino Americans, American Indians, and some Asian Americans and Native Hawaiians or other Pacific Islanders are at particularly high risk for type II diabetes. Type II diabetes is increasingly being diagnosed in children and adolescents.

Gestational diabetes is a form of glucose intolerance that is diagnosed in some women during pregnancy. Gestational diabetes occurs more frequently among African Americans, Hispanic/Latino Americans and American Indians. It is also more common among obese women and women with a family history of diabetes. During pregnancy, gestational diabetes requires treatment to normalize maternal blood glucose levels to avoid complications in the infant. After pregnancy, 5 percent to 10 percent of women with gestational diabetes are found to have type II diabetes. Women who have had gestational diabetes have a 20 percent to 50 percent chance of developing diabetes in the next 5 to 10 years.

Other specific types of diabetes result from specific genetic conditions (such as maturity-onset diabetes of youth), surgery, drugs, malnutrition, infections and other illnesses. Such types of diabetes may account for 1 percent to 5 percent of all diagnosed cases of diabetes.

Over 20 million people or 7 percent of the US population have diabetes, of which only an estimated 14.2 million have been diagnosed with the disease (American Diabetes Association). To survive, people with type I diabetes must have insulin delivered by injections or a pump. Many people with type II diabetes can control their blood glucose by following a careful diet and exercise program, losing excess weight and taking oral medication. Many people with diabetes also need to take medications to control their cholesterol and blood pressure. Diabetes self-management education is an integral component of medical care. Among adults with diagnosed diabetes, 12 percent take both insulin and oral medications, 19 percent take insulin only, 53 percent take oral medications only and 15 percent do not take either insulin or oral medications.

### Diabetes and Wound Healing

About 60 percent to 70 percent of people with diabetes have mild to severe forms of nervous system damage. The results of such damage include impaired sensation or pain in the feet or hands, slowed digestion of food in the stomach, carpal tunnel syndrome and other nerve problems. Severe forms of diabetic wounds are a major contributing cause of lower-extremity amputations.

Diabetes can affect many parts of the body and can lead to serious complications such as blindness, kidney damage and lower-limb amputations. Working together, people with diabetes and their health care providers can reduce the occurrence of these and other diabetes complications by controlling the levels of blood glucose, blood pressure and blood lipids, and by receiving other preventive care practices in a timely manner. Research studies in the United States and abroad have found that improved glycemic control benefits people with either type I or type II diabetes (Collins, N., “Diabetes, Nutrition, and Wound Healing,” Advances in Skin and Wound Care, 291-294, November 2003, which is herein expressly incorporated by reference in its entirety). In general, for every 1 percent reduction in results of glycosylated hemoglobin A1C (A1C) blood tests (e.g., from 8 percent to 7 percent), the risk of developing microvascular diabetic complications (eye, kidney and nerve disease) is reduced by 40 percent. Blood pressure control can reduce cardiovascular disease (heart disease and stroke) by approximately 33 percent to 50 percent and can reduce microvascular disease (eye, kidney and nerve disease) by approximately 33 percent. In general, for every 10 millimeters of mercury (mm Hg) reduction in systolic blood pressure, the risk for any complication related to diabetes is reduced by 12 percent. Improved control of cholesterol or blood lipids (for example, HDL, LDL and triglycerides) can reduce cardiovascular complications by 20 percent to 50 percent, while improving peripheral circulation and the body's ability to fight infection and heal wounds.

U.S. Pat. No. 5,047,249, which is expressly incorporated herein by reference in its entirety, describes compositions and methods for treating skin conditions and promoting wound healing. U.S. Pat. No. 5,487,899, which is expressly incorporated herein by reference in its entirety, describes wound healing compositions. U.S. Pat. No. 6,579,543, which is expressly incorporated herein by reference in its entirety, describes compositions for topical application to the skin.

### SUMMARY OF THE INVENTION

In various embodiments of the invention, delivery or systemic presence of reactive oxygen species (ROS) at the wound site can be used to not only disinfect the wound but directly facilitate the healing process. In an embodiment of the invention, delivery or systemic presence of ROS play a direct role in facilitating angiogenesis by inducing VEGF expression in wound-related cells such as keratinocytes and macrophages. In an embodiment of the invention, a composition consisting of one or two or more compounds of berry extract, chromium, zinc, trans-resveratrol extract, l-arginine, aloe vera, chlorophyll and vitamin C is administered orally or topically to reduce inflammation, improve wound healing, improve innervation of regenerative tissue, improve healing of excisional dermal wounds, improve peripheral circulation, and prevent and/or heal neurodegeneration in a mammal.

In one aspect the mammal is suffering from diabetes mellitus. It is further contemplated that a composition consisting of one or two or more compounds of berry extract, chromium, zinc, trans-resveratrol extract, l-arginine, aloe vera, chlorophyll and vitamin C administered orally or topically to a mammal is used to treat diabetes and one or more of inflammation, slow wound healing, lack of innervation of regenerative tissue, slow healing of excisional wounds, poor peripheral circulation, and neurodegeneration. In a related aspect, the two or more substances are preferably berry extracts and chromium.

In a further embodiment, the invention provides a method for improving wound healing in a mammal, comprising identifying a mammal suffering from or at risk of suffering from wounds; and administering an effective amount of berry extract.

Nutritional factors play a key role in determining wound outcomes in diabetic and non-diabetic wound healing. In an embodiment of the present invention, an optimum nutraceutical formula with one or more compounds of berry extract, chromium, zinc, trans-resveratrol extract, l-arginine, aloe vera, chlorophyll and vitamin C has a synergistic beneficial effect on inflammation, wound healing, peripheral circulation and neuro-regeneration. Wound healing, inflammation, peripheral circulation, and neuro-regeneration can be evaluated orally and topically in both diabetic and non-diabetic mice at various human equivalent doses. l-Arginine at human equivalent doses can be used as a positive control.

Just hours after injury, the wound site recruits inflammatory cells. MCP-1 deficient mice recruit fewer phagocytic macrophages to the injury site. In addition, macrophages that are recruited suffer from compromised functionality. MCP-1 is angiogenic in vivo. At the wound site, macrophages deliver numerous angiogenic products including H₂O₂. This observation, taken together with the result that topical H₂O₂ facilitates dermal wound healing, point towards a clear role of H₂O₂ as a messenger for dermal wound healing (Roy, S. et al., “Dermal wound healing I subject to redox control,” Molecular Therapy 13, 211-220, 2006, which is herein expressly incorporated by reference in its entirety). Over the counter, H₂O₂ is commonly available at strength of 3%. Historically, at such strength H₂O₂ has been clinically used for disinfection of tissues. The use of H₂O₂ to disinfect wounds continues today with a valid concern that at such high doses H₂O₂ may hurt nascent regenerating tissues. Indeed, no beneficial effect of 3% H₂O₂ has been observed.

It is provided that each of the conditions contemplated in the methods of the invention, e.g., reduced inflammation, improved wound healing, improved innervation of regenerative tissue, improved healing of excisional wounds, improved peripheral circulation, and prevention of neurodegeneration in a mammal, may be treated with any of the berry extract, chromium, zinc, trans-resveratrol, l-arginine, aloe vera, chlorophyllin or vitamin C compounds or substances set out below. All of the compounds listed below are described in general terms.

For example, chromium can come from chromium nicotinate, chromium polynicotinate, chromium chloride, chromium picolinate, etc. A preferred form of chromium is niacin-based chromium which is available commercially as CHROMEMATE® (InterHealth Nutraceuticals). Zinc can come from zinc methionine, zinc gluconate, zinc oxide, zinc acetate, etc. One preferred zinc is a methionine-based zinc composition known as OPTIZINC® (InterHealth Nutraceuticals).

Berry extracts are rich in anthocyanins, which are known to act as mild pro-oxidants in wounds and help in wound healing. Anthocyanins also serve as antioxidants, scavengers of free radicals which protect surrounding tissues from phagocytic respiratory bursts and serve as inhibitors of neoplastic processes. They may also be involved in other endogenous wound-healing mechanisms such as improved immune function. In an eight-week study, animals fed OPTIBERRY®, a combination of six standardized berry anthocyanin extracts, and exposed to oxidative stress showed significant whole-body antioxidant protection as compared with control animals. It is contemplated that berry extracts can be from one or more berries selected from the group consisting of blueberry, bilberry, elderberry, cranberry, strawberry, raspberry, blackberry, dewberry, boysenberry, loganberry, youngberries, currant, gooseberry, juniper berry, huckleberry, thimbleberry, blackcap berry, mountain ash berry, salmonberry and wolfberry. Preferred berry extracts for use in the invention include that available commercially as OPTIBERRY® (InterHealth Nutraceuticals). Suitable extracts are described in co-owned U.S. application Ser. No. 10/644,468 published as PCT/US03/29548, the disclosures of which are hereby incorporated by reference. In a related embodiment, the berry extract further comprises anthocyanins.

Chromium is an essential trace mineral required for normal insulin function and glucose homeostasis. Clinical consequences of chromium deficiency include glucose intolerance, hyperlipidemia, metabolic encephalopathy and neropathy. Chromium has been shown to help prevent the buildup of plaque in arteries and improving peripheral circulation by lowering harmful low-density lipoprotein (LDL) cholesterol and increasing beneficial high-density lipoprotein (HDL) cholesterol. It also helps to maintain healthy body weight and normal blood pressure. In addition to blood sugar regulation, insulin is important for protein synthesis in tissue regeneration. During pregnancy, a significant amount of chromium is transported across the placental barrier for the growing fetus, which may be a causative factor for gestational diabetes. Chromium is of particular importance in wound healing as it can alter the immune response by immunostimulatory or immunosuppressive processes as shown by its effects on T and B lymphocytes, macrophages, cytokine production, and the immune response that may induce hypersensitivity reactions. In type 2 diabetics, there are suggestions that chromium supplementation may serve to avoid hyperglycemia and associated protein glycation that contributes to neuropathy and generalized vascular pathology leading to complement fixation compromise and infection. In one aspect, the chromium is selected from the group consisting of chromium nicotinate, chromium polynicotinate, chromium picolinate, chromium chloride, chromium histidinate, and chromium present in yeast.

Zinc is an essential trace mineral and a constituent of the hormone insulin. Zinc is also involved in skin and connective tissue metabolism and in wound healing. Zinc monothionine, a highly bioavailable form of zinc, has been shown to reduce excess levels of free radicals produced by white blood cells, therefore protects the body against free radicals and free radical-induced lipid peroxidation and DNA damages. It is contemplated that the zinc can be selected from the group consisting of zinc methionine, zinc sulfate, zinc polyascorbate, zinc oxide, zinc histidine, zinc gluconate, zinc citrate, zinc acetate, zinc picolinate, zinc alpha-ketoglutarate and zinc aspartate.

trans-Resveratrol is an all-natural phytochemical present in extracts of Polygonum cuspidatum. In vitro and in vivo studies have shown that trans-resveratrol possesses many biological attributes that favor cardio protection, antioxidant activity, modulation of hepatic lipid synthesis and inhibition of platelet aggregation, as well as inhibition of pro-atherogenic eicosanoids by human platelets and neutrophils. trans-Resveratrol has been shown to be a potential chemo preventive agent by inhibiting the cellular events associated with tumor initiation, promotion and progressions. trans-Resveratrol can directly stimulate cell proliferation and differentiation of osteoblasts and decrease tumor growth in vivo. In one aspect, trans-resveratrol is derived from Polygonum cuspidatum. In a related aspect, the trans-xesv eratvol is derived from alternate sources of trans-resveratrol plant or biological extracts, or biological or chemical synthesis.

l-Arginine has been shown to enhance wound breaking strength and collagen synthesis in rodents and humans. Diabetes mellitus, which impairs wound healing, is accompanied by a reduction in nitric oxide at the wound site. The amino acid l-arginine is the only substrate for nitric oxide synthesis. Impaired healing of diabetic wounds can be partially corrected by l-arginine supplementation.

Aloe Vera is known to promote wound healing and is widely effective in treating an assortment of skin diseases. Of the 200 plus species of aloe vera, aloe Barbendisis miller is the most common. It is shown to stimulate repair process and epidermal growth, and to stimulate fibroblast and connective tissue formation, promoting wound repair. Many believe that aloe provides a barrier over the wound to speed the wound healing process. Its active ingredients are anthraquinones, resin, tannins, polysaccharides, prostaglandins and fatty acids. In one aspect, the invention provides methods wherein the aloe vera further comprises aloe Brabendisis miller.

Clorophyllin a and Chlorophyllin b are natural, fat-soluble chlorophylls found in plants. The basic structure of chlorophyll is a porphyrin ring similar to that of heme in hemoglobin. Chlorophyllin is a semi-synthetic mixture of water-soluble sodium copper salts derived from chlorophyll. Chlorophyllin has been used orally as an internal deodorant and topically in the treatment of slow-healing wounds for more than 50 years without any serious side effects. Chlorophyllin can neutralize several physically relevant oxidants in vitro, and limited data from animal studies suggest that chlorophyllin supplementation may decrease oxidative damage induced by chemical carcinogens and radiation. Research in the 1940s indicating that chlorophyllin solutions slowed the growth of certain anaerobic bacteria in the test tube and accelerated the healing of experimental wounds in animals led to the use of topical chlorophyllin solutions and ointments in the treatment of persistent open wounds in humans. During the late 1940s and 1950s, a series of largely uncontrolled studies in patients with slow-healing wounds, such as vascular ulcers and pressure (decubitus) ulcers, reported that the application of topical chlorophyllin promoted healing more effectively than other commonly used treatments. In the late 1950s, chlorophyllin was added to papain and urea-containing ointments used for the chemical debridement of wounds in order to reduce local inflammation, promote healing and control odor. Chlorophyllin-containing papain/urea ointments are still available in the US by prescription. In one embodiment of the methods of the invention, the chlorophyllin can be selected from water soluble chlorophyllin, sodium chlorophyllin, copper chlorophyllin, fat soluble chlorophyll A and fat soluble chlorophyll B.

Vitamin C is necessary for a normal response to physiological stressors, with the need increasing during times of injury or stress. Wounds, including trauma, burns and major surgery have been correlated with a decrease in plasma vitamin C. A lack of vitamin C results in impaired wound healing, as well as breakdown of previously healed wounds. During the inflammatory stage of wound healing, and to a lesser degree throughout the healing process, production of ROS required to kill bacteria can exceed its usefulness and cause local collateral damage to tissues and organs throughout the body. As an antioxidant, vitamin C is useful in controlling excess ROS. Studies have shown that vitamin C deficiency results in decreased tensile strength and collagen production, as well as altered angiogenesis, reversed by vitamin C supplementation. Vitamin C also plays a role in the immune response and antibacterial activity required in fighting infection. Infections resulting from impaired immunity is one of the most commonly encountered and clinically significant impediments to wound healing. Vitamin C has been shown to improve immune function in humans. It is contemplated that the vitamin C is be selected from the group consisting of ascorbic acid, esterfied ascorbic acid and mineral salts of ascorbic acid including calcium ascorbate, magnesium ascorbate, potassium ascorbate and zinc ascorbate.

In a further embodiment, the invention provides a composition for administration to reduce inflammation, improve wound healing, improve innervation of regenerative tissue. improve healing of excisional wounds, improve peripheral circulation, and prevent neurodegeneration in a mammal comprising an amount of berry extract between: a lower limit of approximately 3 mg human equivalency dosage (HED) per day; and an upper limit of approximately 500 mg HED per day; an amount of chromium between: a lower limit of approximately 10 μg HED per day; and an upper limit of approximately 1000 μg HED per day; an amount of zinc between: a lower limit of approximately 1.5 mg HED per day; and an upper limit of approximately 75 mg HED per day; an amount of trans-resveratrol between: a lower limit of approximately 0.1 mg HED per day; and an upper limit of approximately 50 mg HED per day; an amount of l-arginine between: a lower limit of approximately 50 mg HED per day; and an upper limit of approximately 5000 mg HED per day; an amount of chlorophyllin between: a lower limit of approximately 10 mg HED per day and an upper limit of approximately 300 mg HED per day; an amount of vitamin C between: a lower limit of approximately 10 mg HED per day and an upper limit of approximately 10,000 mg HED per day. In one aspect the tarns-resveratrol may be an amount of Polygonum cuspidatum extract between: a lower limit of approximately 1.5 mg HED per day; and an upper limit of approximately 100 mg HED per day.

In a related aspect, the invention provides a composition for topical application to reduce inflammation, improve wound healing, improve innervation of regenerative tissue, improve healing of excisional wounds, improve peripheral circulation, and prevent neurodegeneration in a mammal. In one embodiment the composition above is for topical application and further comprises: an amount of aloe Barbendisis miller between: a lower limit of approximately 0.01% (by weight) and an upper limit of approximately 10% (by weight). In a related embodiment, the composition for topical application comprises an amount of berry extract between: a lower limit of approximately 0.01% (by weight)and an upper limit of approximately 10% (by weight); an amount of chromium between: a lower limit of approximately 0.01% (by weight) and an upper limit of approximately 10% (by weight); an amount of zinc between: a lower limit of approximately 0.01% (by weight) and an upper limit of approximately 10% (by weight); an amount of trans-resveratrol between: a lower limit of approximately 0.01% (by weight) and an upper limit of approximately 10% (by weight); an amount of l-arginine between: a lower limit of approximately 0.01% (by weight) and an upper limit of approximately 10% (by weight); an amount of aloe Barbendisis miller between: a lower limit of approximately 0.01% (by weight) and an upper limit of approximately 10% (by weight); an amount of chlorophyllin between: a lower limit of approximately 0.01% (by weight) and an upper limit of approximately 5% (by weight); an amount of vitamin C between: a lower limit of approximately 0.01% (by weight) and an upper limit of approximately 10% (by weight). In one aspect, the trans-resveratrol may be an amount of Polygonum cuspidatum extract between: a lower limit of approximately 0.01% (by weight) and an upper limit of approximately 10% (by weight);

### BRIEF DESCRIPTION OF DRAWINGS

FIG. 1A shows a plot of wound area as a % of initial wound for diabetic versus non-diabetic mice;

FIG. 1B shows hematoxylin and eosin staining of the wounds on diabetic mouse day 3, post wounding;

FIG. 1C shows hematoxylin and eosin staining of the wounds on non-diabetic mouse day 3, post wounding;

FIG. 1D shows TUNEL staining of the wounds on diabetic mouse day 3, post wounding;

FIG. 1E shows plot of apoptotic cells for diabetic (NOR) versus non-diabetic (NOD) mice; and

FIG. 1F shows individual macrophage stained with anti-F4/80 coupled with FITC and counterstained with blue.

FIG. 2 shows a comparison of wound healing in mice administered Polygonum cuspidatum extract (standardized to 50% trans-resveratrol) commercially available as Protykin® (InterHealth Nutraceuticals);

FIG. 3 shows a comparison of wound healing in mice administered a methionine-bound zinc composition commercially available as OptiZinc® (InterHealth Nutraceuticals);

FIG. 4 shows a comparison of wound healing in mice administered a multiple berry anthocyanin extract composition commercially available as OptiBerry® (InterHealth Nutraceuticals);

FIG. 5 shows a comparison of wound healing in mice administered a niacin-bound chromium commercially available as ChromeMate® (InterHealth Nutraceuticals);

FIG. 6 shows a comparison of blood glucose levels compared to placebo-gavaged control mice for a Polygonum cuspidatum extract (standardized to 50% trans-resveratrol); a methionine-bound zinc composition; a multiple berry anthocyanin extract; niacin-bound chromium and a mixture of equal parts by weight of the four single ingredients;

FIG. 7 shows a comparison of cholesterol levels compared to placebo-gavaged control mice for a Polygonum cuspidatum extract (standardized to 50% trans-resveratrol); a methionine-bound zinc composition; a multiple berry anthocyanin extract; niacin-bound chromium and a mixture of equal parts by weight of the four single ingredients;

FIG. 8 shows a comparison of HDL cholesterol levels compared to placebo-gavaged control mice for a Polygonum cuspidatum extract (standardized to 50% trans-resveratrol); a methionine-bound zinc composition; a multiple berry anthocyanin extract; niacin-bound chromium and a mixture of equal parts by weight of the four single ingredients;

FIG. 9 shows a comparison of triglyceride levels compared to placebo-gavaged control mice for a Polygonum cuspidatum extract (standardized to 50% trans-resveratrol); a methionine-bound zinc composition; a multiple berry anthocyanin extract; niacin-bound chromium and a mixture of equal parts by weight of the four single ingredients (Mix 1);

FIG. 10 shows a comparison of calculated LDL levels compared to placebo-gavaged control mice for a Polygonum cuspidatum extract (standardized to 50% trans-resveratrol); a methionine-bound zinc composition; a multiple berry anthocyanin extract; niacin-bound chromium and a mixture of equal parts by weight of the four single ingredients;

FIG. 11 shows a comparison of total cholesterol/HDL levels compared to placebo-gavaged control mice for a Polygonum cuspidatum extract (standardized to 50% trans-resveratrol); a methionine-bound zinc composition; a multiple berry anthocyanin extract; niacin-bound chromium and a mixture of equal parts by weight of the four single ingredients;

FIG. 12 shows a comparison of wound healing in mice administered a mixture of equal parts by weight of a Polygonum cuspidatum extract (standardized to 50% trans-resveratrol); a methionine-bound zinc composition; a multiple berry anthocyanin extract; and niacin-bound chromium (Mix 1);

FIG. 13 shows a comparison of wound healing in mice administered a mixture of equal parts by weight of a multiple berry anthocyanin extract; and niacin-bound chromium (Mix 2); and

FIG. 14 shows a comparison of wound healing in mice administered a mixture at a 4:4:1:1 ratio by weight of a Polygonum cuspidatum extract (standardized to 50% trans-resveratrol); a methionine-bound zinc composition; a multiple berry anthocyanin extract; and niacin-bound chromium (Mix 3).

### DETAILED DESCRIPTION OF THE INVENTION

### Phase I: In Vivo Dose-Response Study

The effect of one or more compounds of berry extract, chromium, zinc, trans-resveratrol, l-arginine, aloe vera, and chlorophyllin (vitamin C is produced naturally by mice, but not in humans) on wound healing and inflammation can be evaluated orally and topically in both diabetic and non-diabetic mice (six animals per group) at various Human Equivalent Doses Oral HED=(oral animal dosage)×(Human weight/animal weight)^{1/3}, topical HED=topical animal dosage. l-Arginine at human equivalent doses can be used as a positive control.

The average weight for male adults in the United States is about 76-83 Kg. The average weight for female adults in the United States is about 54-64 Kg. The average weight for mice is 20 gm. Thus, the conversion equations for oral Human Equivalent Dosage to mice dosages are approximately oral HED=15×oral mice dosage. Topical HED=topical animal dosage.

### Test Groups:

### Group A—Diabetic Oral (Taken 1-3 Months Before Wounding)

### Diabetic Mice Control

Berry extract at 3, 10, 50, 100, 250 and 500 mg human equivalency dosages; Elemental chromium at 10, 50, 100, 200, 400 and 1000 μg human equivalency dosages; Elemental zinc at 1.5, 7.5, 15, 25, 50 and 75 mg human equivalency dosages; Polygonum cuspidatum extract (standardized to 50% trans-resveratrol) at 1, 5, 10, 25, 50 and 100 mg human equivalency dosages;

l-Arginine at 10, 100, 500, 1,000, 5,000 and 10,000 mg human equivalency dosages;

Chlorophyllin (sodium copper salts of chlorophyll) at 10, 50, 100, 150, 200, 250 and 300 mg human equivalency dosages.

### Group B—Non-Diabetic Oral (Taken 1-3 Months Before Wounding)

### Non-Diabetic Mice Control

Berry extract at 3, 10, 50, 100, 250 and 500 mg human equivalency dosages; Elemental chromium at 10, 50, 100, 200, 400 and 1000 μg human equivalency dosages; Elemental zinc at 1.5, 7.5, 15, 25, 500 and 75 mg human equivalency dosages;

Polygonum cuspidatum extract (standardized to 50% trans-resveratrol) at 1, 5, 10, 25, 50 and 100 mg human equivalency dosages

l-Arginine at 10, 100, 500, 1,000, 5,000 and 10,000 mg human equivalency dosages; Chlorophyllin (sodium copper salts of chlorophyll) at 10, 50, 100,150, 200, 250 and 300 mg human equivalency dosages.

Initial pilot studies can be conducted in all treatment groups using the highest oral doses. The objective of experiments proposed under this section is to characterize wound healing in type II diabetes. Specifically, studies can be focused on whether these mice have a prolonged inflammatory phase, as has been suggested to be a leading cause of most non-healed wounds in diabetics. For example, mice can be fed a diet consisting of one or more compounds of 33 mg berry extract, 67 μg elemental chromium, 5 mg elemental zinc, 16.7 mg Polygonum cuspidatum extract, 67 mg l-arginine, and 20 mg chlorophyllin for 4-12 weeks prior to inflicting the wound. Alternatively, mice can be topically treated twice daily for 30 days after wounding using a cream or gel on their wound comprising one or more of 0.01 wt % to 10 wt % of chromium, 0.01 wt % to 10 wt % of zinc, 0.01 wt % to 10 wt % of berry extracts, 0.01 wt % to 10 wt % of Polygonum cuspidatum extract, 0.01 wt % to 10 wt % of L-arginine, 0.01 wt % to 10 wt % of aloe Barbendisis miller, and 0.01 wt % to 5 wt % of chlorophyllin.

Blood glucose levels can be monitored weekly by obtaining blood from tail-nick and using a glucometer (Elite system, Bayer). The animals can be used after 3-4 weeks of blood glucose reaching the 250 mg/dL level. Wound healing can be assessed according to the following criteria:

### A. Wound Closure.

For wound closure and contraction studies, two full thickness (5×10 mm) excisional wounds can be placed on the dorsum of male 8-10 wks db/db (Chen, H. et al., “Evidence that the Diabetes Gene Encodes the Leptin Receptor: Identification of a mutation in the Leptin Receptor Gene in db/db Mice,” Cell, 84:491-495, 1996, which is herein expressly incorporated by reference in its entirety) as well as the corresponding (age and sex matched) control (heterozygous, db/+) mice. Wound area can be measured by imaging wounds every alternate day post wounding using a digital camera (Canon Powershot G6) and a reference scale. Wound area from the images can be calculated using WoundMatrix™ software.

### B. Histology

For histology, two 3 mm full thickness punch biopsy wounds can be made. The entire wound can be harvested using a 6 mm punch biopsy. One of the two wounds can be formalin fixed and paraffin embedded, while the other one can be collected frozen in OCT followed by sectioning using a microtome (or crymicrotome for frozen sections). To visualize general wound architecture, the sections can be stained with hematoxylin-eosin (HE) and Masson Trichrome.

### C. Inflammation.

The inflammatory phase can be characterized by quantifying the number of mE1 and neutrophil in histological sections on specific days (0, 1, 3, 5, 7d) following wounding. The cells can be detected using standard immuno-staining protocol and the following detection antibody (in parentheses): mU (MOMA-2, F4/80, Mac1) and neutrophils (Anti-Ly-6G, anti-neutrophil). The number of mil or neutrophils within a section can be enumerated on five visual fields under high magnification (40×). Laser Doppler wound blood flow imaging. Establishment of proper blood flow is a marker of successful regeneration of tissue at the wound site. Wound blood flow imaging can be performed by Doppler blood flow imager (Moor Instruments) that offers a high spatial resolution. Expected results, potential problems, and solutions. Based on previous studies, it is anticipated that diabetics exhibit an impaired wound closure as compared to their corresponding controls. Choice of proper control with each model is critical for proper interpretation of the data obtained. Age and gender-matched heterozygous (dbl+) mice as control. The heterozygotes show normal body weight, blood glucose and plasma insulin. Because of compromised leukocyte function, diabetic mice are susceptible to infections. Routine checks for the presence of microbial flora in wounds of diabetic and non-diabetic mice can be carried out to assess that the observed differences are not merely due to difference in wound microflora.

Migration/Transportation of Berry Extract, Chromium, Zinc, Polygonum cuspidatum Extract, Aloe Vera and Chlorophyllin

Chlorophyllin or l-arginine migration/transportation to the subcutaneous or dermal tissue following oral administration can be assessed at 30 and 90 days of treatment, and quantified by HPLC. Based on these results, two additional oral doses can be selected from the above list for all treatment groups and migration/transportation of berry extract, chromium, zinc, trans-resveratrol, l-arginine, aloe vera and chlorophyllin to the subcutaneous or dermal tissue can be quantified by HPLC at 30 and 90 days of treatment. All the Test Parameters (itemized below) can be evaluated in all groups.

Based on the results obtained in Groups A and B, and the available concentrations of similar active components in the creme/topical formulations in the marketplace, 3 doses are selected for topical application for each product. A suitable vehicle is a hydrophilic based jelly. Alternative vehicles to a hydrophilic based jelly are also within the spirit of this invention. All the Test Parameters (itemized below) can be evaluated in all groups.

### Group C—Diabetic Topical (Applied Twice Daily up to 30 Days After Wounding):

### Diabetic Mice Control

Berry extract: 0.01-10% weight percent;

Elemental chromium: 0.01-10% weight percent;

Elemental zinc: 0.01-10% weight percent;

Polygonum cuspidatum extract (standardized to 50% trans-resveratrol): 0.01-10% weight percent;

l-Arginine: 0.01-10% weight percent;

Aloe Barbendisis miller: 0.01-10% weight percent;

Chlorophyllin (sodium copper salts of chlorophyll): 0.01-5% weight percent

### Group D—Non-Diabetic Topical (Applied Twice Daily for 30 Days After Wounding):

### Non-Diabetic Mice Control

Berry extract: 0.01-10% weight percent;

Elemental chromium: 0.01-10% weight percent;

Elemental zinc: 0.01-10% weight percent;

Polygonum cuspidatum extract (standardized to 50% trans-resveratrol): 0.01-10% weight percent;

l-Arginine: 0.01-10% weight percent;

Aloe Barbendisis miller: 0.01-10% weight percent;

Chlorophyllin (sodium copper salts of chlorophyll): 0.01-5% weight percent

### Test Parameters:

### 1. Wound Healing:

a. Acceleration of wound healing (before and after picture)
b. Angiogenesis
c. Immuno-histochemistry/scarring
d. Glutathione
e. Inflammation markers (TNFα, Cytokines)
f. Healing related genes
g. Nerve damage markers
h. Glucose/insulin markers

### Phase II: In Vivo Combinations

Based on Phase I results, as well as ingredient cost considerations, various combinations and concentrations of ingredients can be formulated and re-tested in both diabetic and non-diabetic mice (six animals per group) according to the same test parameters in Phase I to determine the safest and most effective combinations in vivo.

### Formulations:

Formulations designed for oral administration shall consist of two or more of the following compounds: berry extract, chromium, zinc, Polygonum cuspidatum extract (standardized to 50% trans-resveratrol), l-arginine and chlorophyllin.

Formulations can be designed for topical application shall consist of two or more of the following compounds: zinc, chromium, berry extract, Polygonum cuspidatum extract (standardized to 50% trans-resveratrol), aloe vera, l-arginine and chlorophyllin.

### EXAMPLE 1

Wound healing is impaired in type I diabetic mice. To determine the effect of the compositions described herein on wound healing in diabetic mammals, two full-thickness excisional wounds were placed on the dorsal skin of diabetic NOD/LtJ mice and matched control non-obese non-diabetic NOR/LtJ mice (12-15 wks, 5×10 mm wounds).

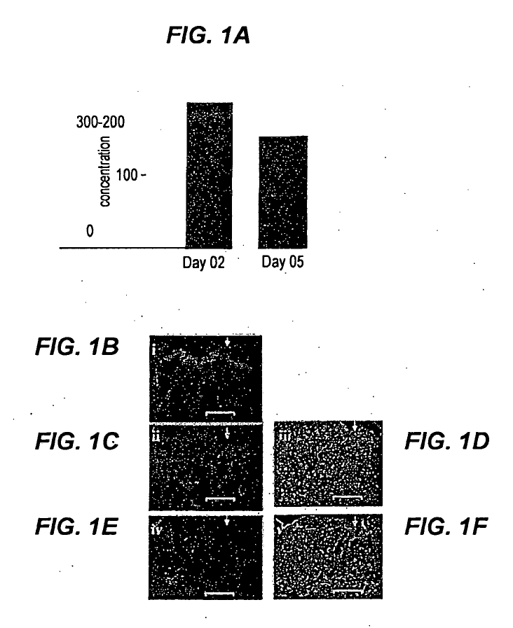

Results are shown in FIG. 1 A. Wound area is shown as % of area of initial wound. Data are shown as mean±SD (n=4)*, p<0.05 versus corresponding non-diabetic control mice. As shown in FIG. 1B-FIG. 1C, histological analyses using hematoxylin and eosin staining of the wounds on day 3, post wounding, clearly demonstrated increased cellularity in NOD wounds versus NOR wounds (Scale bar=100 μm).

To visualize dead cells in the wound tissue, deoxynucleotidyl transferase-mediated dUTP nick end labeling (TUNEL) staining was optimized. Stained images show (FIG. 1D) a positive control that was generated by treating the tissue section treated with proteinase K and nuclease. TUNEL positive apoptotic cells can be seen with black nuclear stain (Scale bar=100 μm) (FIG. 1E). Results of TUNEL positive cell scoring show that the number of dead cells in the wound tissue was higher in wound tissue (d 3 post wounding). Data shown are mean±SD (n=3), p<0.05 compared to NOR animals.

PVA sponges were implanted subcutaneously on the back of NOD and NOR animals. The sponges were harvested on d5 of implantation and the cells were sorted through magnetic sorting to isolate pure macrophage (mφ, F4 180 positive) population from the PVA sponge cell suspension. Mφ suspension was then cytospun. Individual macrophage stained using anti-F4/80 coupled with FITC (green) and counterstained using DAPI (blue) (Scale bar=10 μm) (FIG. 1F). The F4/80 staining of wound mφ cytospin showed over 98% pure mφ population after the magnetic sorting thus establishing efficacy of this technique.

Mice homozygous (BKS.Cg-m+1+Lepr′) for spontaneous mutation of the leptin receptor (Lepr″) become identifiably obese around 3 to 4 weeks of age. Elevation of blood sugar is evident at 4 to 8 weeks after birth.

Blood glucose levels were monitored weekly by obtaining blood from tail-nick and using a glucometer (Elite system, Bayer). The animals were analyzed 3-4 weeks after blood glucose reached the 250 mg/dL level.

These results suggest that future studies can be focused on whether these mice have a prolonged inflammatory phase, as has been suggested to be a leading cause of most non healed wounds in diabetics.

### Wound Healing in Diabetic Mice

### Wound Healing can be Assessed According to the Following Criteria:

### A. Wound Closure.

For wound closure/contraction studies, two full thickness (5×10 mm) excisional wounds can be placed on the dorsum of male 8-10 wks db/db as well as the corresponding (age and sex matched) control (heterozygous, db/+) mice. Wound area can be measured by imaging wounds every alternate day post wounding using a digital camera (Canon Powershot G6) and a reference scale. Wound area from the images can be calculated using WOUNDMATRIX™ software.

### B. Histology.

For histology, two 3 mm full thickness punch biopsy wounds can be made. The entire wound can be harvested using a 6 mm punch biopsy. One of the two wounds can be formalin fixed and paraffin embedded while the other one can be collected frozen in OCT followed by sectioning using a microtome (or crymicrotome for frozen sections). To visualize general wound architecture, the sections can be stained with hematoxylin-eosin (HE) and Masson Trichrome.

### C. Inflammation.

The inflammatory phase can be characterized by quantifying the number of mφ and neutrophil cells in histological sections on specific days (0, 1, 3, 5, 7 day) following wounding. The cells can be detected using standard immuno-staining protocols and the following detection antibody: mφ (MOMA-2, F4/80, Mac1) and neutrophils (Anti-Ly-6G, anti-neutrophil). The number of mφ or neutrophils within a section can be enumerated on five visual fields under high magnification (40×).

### D. Laser Doppler Wound Blood Flow Imaging.

Establishment of proper blood flow is a marker of successful regeneration of tissue at the wound site. Wound blood flow imaging can be performed by Doppler blood flow imager (Moor Instruments) that offers a high spatial resolution.

### Expected Results, Potential Problems and Solutions

Based on previous studies, it is anticipated that diabetic patients show an impaired wound closure as compared to their corresponding controls. Choice of proper control with each model is critical for proper interpretation of the data obtained. Age and gender-matched heterozygous (dbl+) mice are used as control. The heterozygotes show normal body weight, blood glucose, and plasma insulin. Because of compromised leukocyte function, diabetic mice are susceptible to infections. The presence of microbial flora in wounds of diabetic and non-diabetic mice is checked to assess that the observed differences are not merely due to difference in wound microflora.

### Wound Models

### A. Secondary-Intention Excisional Dermal Wound Model.

Wounding of mice is performed after anesthetized with isoflurane inhalation. For wound contraction (closure) and biochemical (RNA, protein etc.) studies, two 5×10 mm full-thickness (skin and panniculus carnosus) excisional wounds are placed on the dorsal skin (shaved and cleaned using betadine), equidistant from the midline and adjacent to the four limbs. The wounds are allowed to dry to form a scab. For wound histology studies, two 3 mm full-thickness (using a biopsy punch; skin and panniculus camosus) excisional wounds are placed on the dorsal skin (shaved and cleaned using betadine), equidistant from the midline and adjacent to the four limbs. The wounds are allowed to dry to form a scab. On the specified day of harvest, the entire wounds are harvested using a 6 mm biopsy punch. One of the wounds can be formalin fixed and paraffin embedded for histology purposes. The other wounds are collected in OCT for frozen sectioning and histology. Microbial flora in wounds is determined routinely to determine that the observed differences are not merely due to difference in wound microflora. To minimize wound infections, all mice for the experiments are housed in a sterile facility.

### B. “Hunt/Schilling” Wire Mesh Cylinder for Wound Fluid Collection.

This method is used to collect wound fluid during the course of healing. The implantation of wire mesh cylinder (stainless steel; 2.5 cm length and 0.8 cm diameter) and wound fluid harvest is performed.

### C. Histologic Evaluation.

Formalin-fixed paraffin embedded wound tissue blocks are sectioned using a microtome (4 μm thick). For histological evaluations, the sections are stained with hematoxylin and eosin (HE) as well as for Masson Trichrome staining. Immunostaining: Formalin-fixed paraffin-embedded or acetone fixed frozen-tissue sections or cytospins are labeled and detected with appropriate primary and secondary (HRP or fluorochrome-tagged) antibody. HRP tagged secondary antibody can be developed using DAB as substrate (brown color).

### D. Laser Doppler Wound Blood Flow Imaging.

Establishment of proper blood flow is a marker of successful regeneration of tissue at the wound site. Wound blood flow imaging can be performed by Doppler blood flow imager (Moor Instruments) that offers a high spatial resolution.

### E. Assessment of Microbial (Bacterial) Growth in Wounds.

Routine analyses of wound swabs normally involve the use of non-selective (e.g., LB and blood agar) and selective (e.g., MacConkey) agar plates. For the proposed studies related to wound microbiology, LB agar plates are used. For gram-negative bacteria, MacConkey agar plates are used.

### F. Superficial Bacterial Load:

The entire surface of the wound can be swabbed for 20 sec using an alginate-tipped applicator. The tip of the swab is broken off and placed into sterile tube containing saline. Serial dilution of quantitative swabs is performed and plated on sterile LB agar medium. All plated specimens are incubated under aerobic conditions at 37° C. After 24 hours, the plates are visually inspected and colonies of bacteria counted. Colony forming units (CFU) are then utilized to determine the total bacterial count on each plate 6. Deep tissue bacterial load: The superficial eschar tissue is removed. Wound bed tissue underneath eschar is biopsied aseptically, weighed, homogenized, serially diluted and cultured on LB agar plates as described above. Quantitative assessment of bacterial load is determined by counting the number of colonies on each plate.

G. mRNA Quantitation.

Total RNA is extracted using Trizol (Invitrogen) and RNAeasy kit (Qiagen). Quantitative or real-time PCR (Taqman or Sybr Green) approach is used for mRNA quantification.

### H. Synergism

A synergistic effect is when the effect of the sum of the entities is more than the effect of any one entity. In this field, where a multitude of factors work significantly more potently than any one factor alone, it is considered unfeasible to establish the effect of all the separate entities alone. In the present invention, it is not required that the effect of all separate entities be established and then that the effect observed is greater than the sum of the individual effects observed.

### EXAMPLE 2

In order to determine the effects of oral and topical administration of nutritional supplements of the invention on wound healing in diabetic mice, mice having the Leprdb/Leprdb mutation were administered nutraceutical formulations of the formulations of the present invention either orally or topically, and the improvement in wound healing assessed.

Leprdb mice were maintained on a normal laboratory diet and administered nutritional supplements as described in the detailed description, e.g., via supplements by oral gavage (10 mg/kg body weight; 8 weeks hand gavage) or topically (10 μl on day 0, 1, and 2; stock 3 mg/ml solution). Nutritional supplements administered include a Polygonum cuspidatum extract (standardized to 50% trans-resvevatrol) commercially available as PROTYKIN® (InterHealth Nutraceuticals), a methionine-bound zinc composition commercially available as OPTIZINC® (InterHealth Nutraceuticals), a multiple berry anthocyanin extract commercially available as OPTIBERRY® (InterHealth Nutraceuticals), and a niacin-bound chromium commercially available as CHROMEMATE® (InterHealth Nutraceuticals). Mice were injured by an excisional wound as described previously after 2 months of oral administration of supplements or at the time of topical administration.

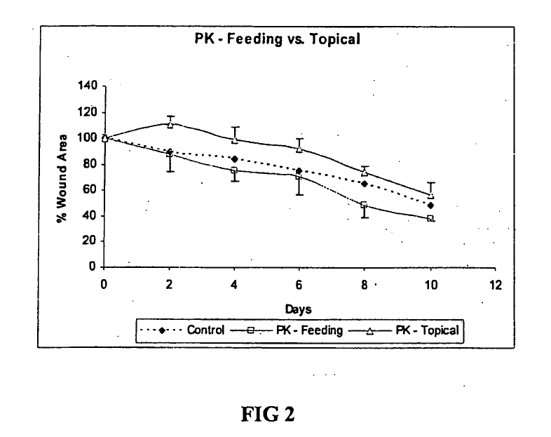

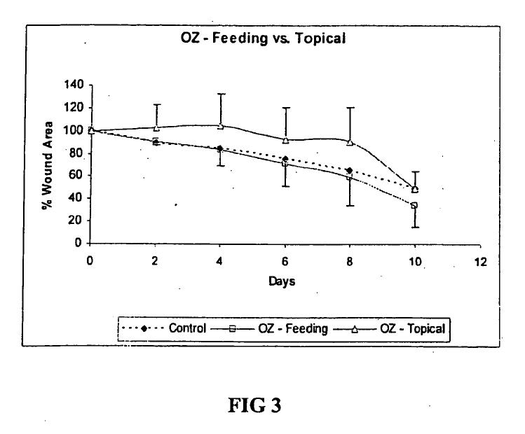

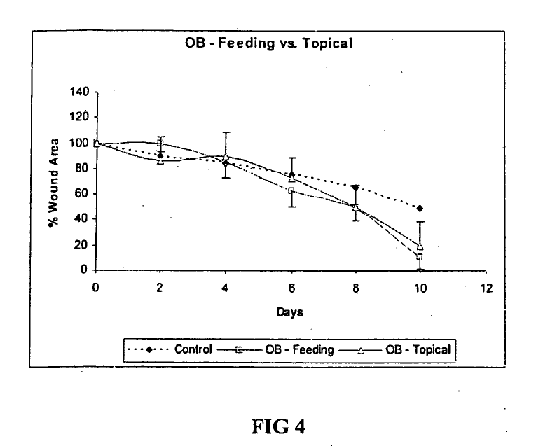

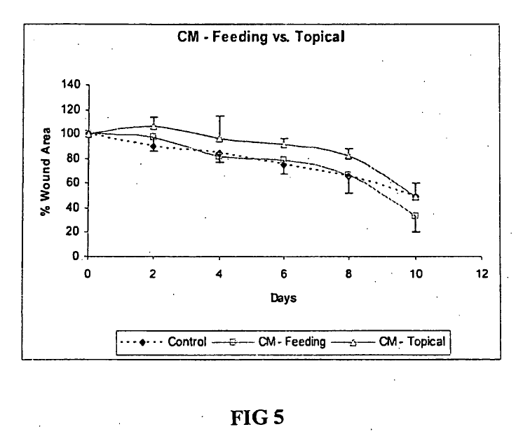

Experimental results show that PROTYKIN® feeding improved wound closure of diabetic mice whereas topical application of the supplement caused dilation at the wound site and did not result in improvement (FIG. 2). Feeding OPTIZINC® resulted in improved wound closure in diabetic mice compared to controls (FIG. 3) but also aggravated the wound site causing dilation at the site when applied topically. Oral administration of the OPTIBERRY® formulation significantly improved wound closure in the affected mice (FIG. 4), and did not have any negative effects when applied topically. Administration of the chromium formulation CHROMEMATE® appeared to not affect wound closure in diabetic mice when administered either orally or topically (FIG. 5).

These results demonstrate that administration of the OPTIBERRY® formulation significantly improved wound healing when administered both orally and topically and may provide a useful tool to combat a common side-effect of diabetes in human patients.

### EXAMPLE 3

In addition to determining the effects of nutritional supplements on external healing in diabetic patients, the effects of the formulations of blood glucose levels and lipid levels were also assessed. Mice were treated with nutritional supplements as described in Example 2 and blood glucose levels and lipid profiles assessed as described in Rink et al., Physiol Genomics 27:370-9, 2006.

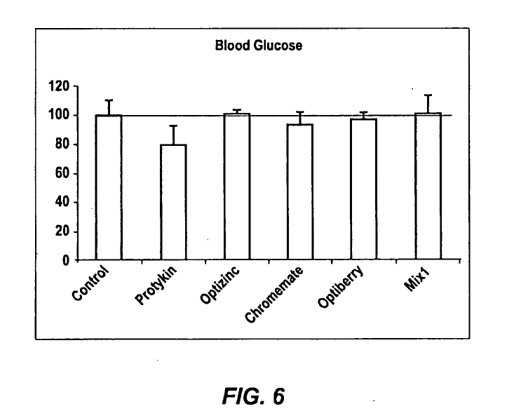

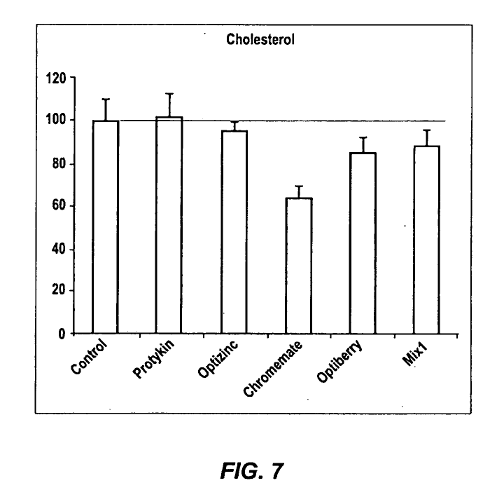

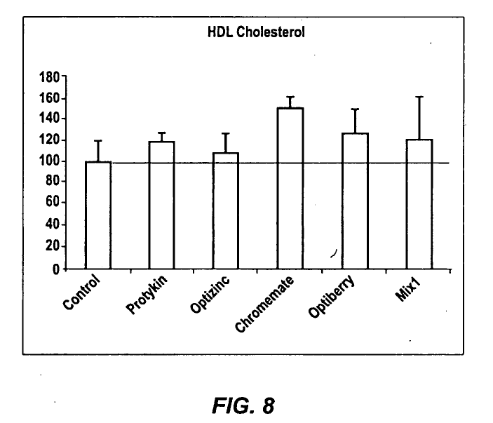

Treatment with PROTYKIN® tended to lower blood glucose levels compared to placebo controls, while OPTIZINC®, CHROMEMATE®, and OPTIBERRY® had no effect on blood glucose levels (FIG. 6). Measurement of cholesterol levels in treated diabetic mice showed that CHROMEMATE® lowered blood cholesterol by approximately 35% (FIG. 7) while OPTIBERRY® lowered cholesterol levels by approximately 15-20%. Additionally CHROMEMATE® increased blood levels of HDL cholesterol by approximately 50% and OPTIBERRY® increased HDL levels by approximately 20% (FIG. 8). PROTYKIN also increased HDL by approximately 20%.

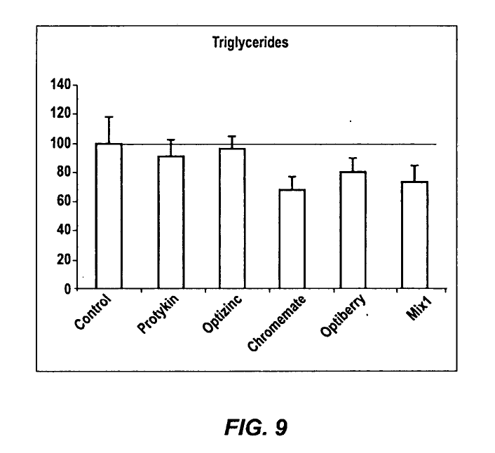

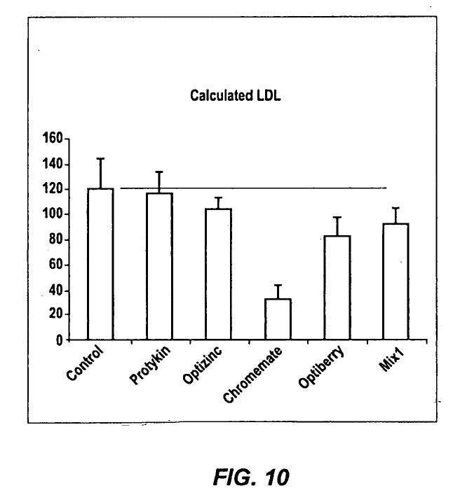

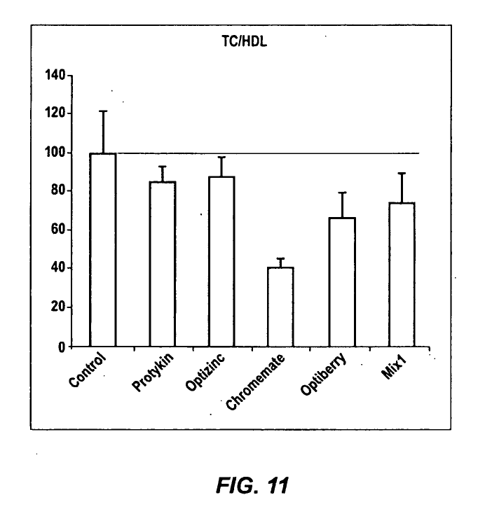

Triglycerides and LDL levels were also assessed in diabetic and control mice. Treatment with CHROMEMATE® decreased triglycerides by approximately 35% (FIG. 9) and treatment with OPTIBERRY® decreased triglyceride levels by approximately 20%. LDL levels were decreased approximately 80% by administration of CHROMEMATE® (FIG. 10) and by approximately 40% by OPTIBERRY®. Administration of the CHROMEMATE® formulation also decreased the total ratio of cholesterol/HDL by approximately 60% (FIG. 11) while treatment with OPTIBERRY® decreased the cholesterol/HDL ratio in diabetic mice by approximately 40%.

Histological assessment of treated mice was performed as described above. OPTIBERRY® treated Leprdb mice were assessed for macrophage infiltration into the wound site at day 10 post excisional wound. Histological sections showed that OPTIBERRY® therapy decreased the distance between skin layers in wounded mice and improved wound healing in these animals. Histological evidence also showed that macrophage infiltration at the wound site at day three was increased slightly as a result of OPTIBERRY® treatment.

These results demonstrate that in addition to improving the speed of wound healing in diabetic animals, the nutritional supplements can also improve blood glucose and cholesterol levels in treated animals.

### EXAMPLE 4

### Administration of Mixed Supplement Formulations

In addition to administration of the supplements individually, treatment of supplements in various combinations and ratios were also assessed for their effect on wound healing. Three different formulations were assessed for the effects on wound closure. Mix 1 was a mix having a 1::1:1:1 ratio of PROTYKIN®, CHROMEMATE®, OPTIBERRY® and OPTIZINC®. Mix 2 was a mixture comprising a 1:1 ratio of OPTIBERRY® and CHROMEMATE®. Mix 3 was a mixture comprising a 4:4:1:1 ratio of OPTIBERRY®, CHROMEMATE®, PROTYKIN® and OPTIZINC®, respectively.

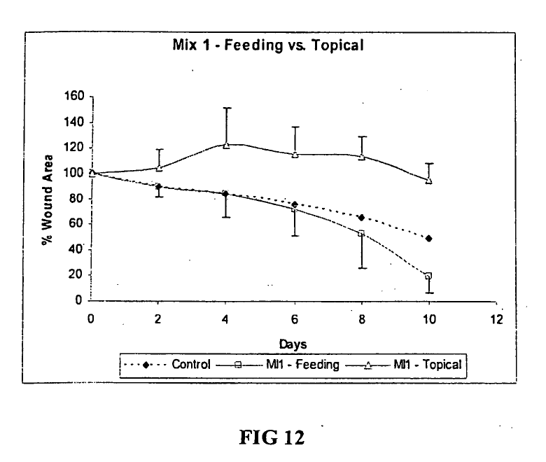

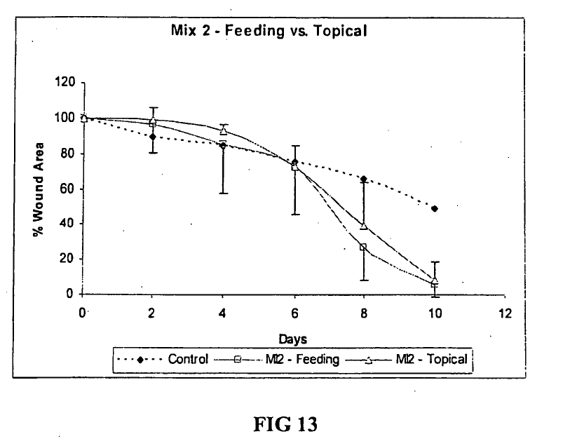

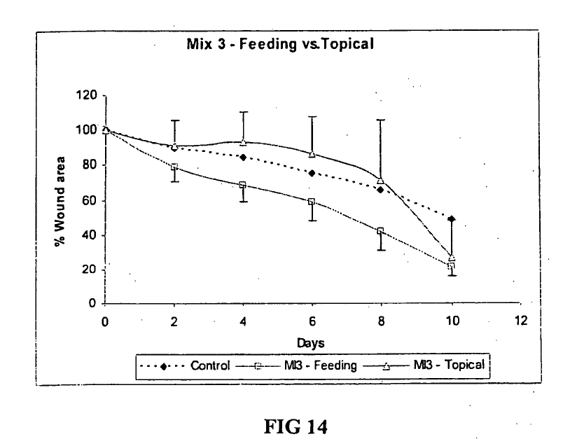

The mixed formulations were administered either orally or topically as described in Example 2 (orally, 10 mg/kg body weight; 8 weeks hand gavage or topically, 10 μl on day 0,1, and 2; stock 3 mg/ml solution) and the effect on wound closure assessed. The data show that feeding Mix 1 improved wound closure while topical administration adversely affected wound closure in diabetic animals (FIG. 12). Mix 2 showed clear improvement in wound closure when the mixture was given orally, whereas topical application showed no effect on wound closure (FIG. 13). Oral administration of Mix 3 (FIG. 14) demonstrated a slight improvement in wound closure and topical application showed initial adverse effects but these affects cleared by day 9 post wound.

Additionally, measurement of glucose and cholesterol levels demonstrated that administration of Mix 1 exhibited similar effects as the OPTIBERRY® alone treatment, lowering blood cholesterol levels, triglycerides, LDL levels, and the cholesterol/HDL ratio in diabetic animals, while slightly increasing HDL levels (FIGS. 6-11).

These results demonstrate that mixtures of nutritional supplements are as effective, and perhaps more effective, at speeding wound healing than a nutritional supplement given individually.

Numerous modifications and variations in the invention as set forth in the above illustrative examples are expected to occur to those skilled in the art. Consequently only such limitations as appear in the appended claims should be placed on the invention

1. A method for preventing or reducing inflammation in a mammal, the method comprising:
identifying a mammal suffering from or at risk of suffering from inflammation; and
administering an effective amount of two or more of the following substances: berry extract, chromium, zinc, trans-resveratrol, l-arginine, chlorophyll, vitamin C, and aloe vera, wherein the administering prevents or reduces the inflammation.

2. A method for improving wound healing in a mammal, the method comprising:
identifying a mammal suffering from or at risk of suffering from wounds: and
administering an effective amount of two or more of the following substances: berry extract, chromium, zinc, trans-resveratrol, l-arginine, chlorophyll, vitamin C, and aloe vera, wherein the administering improves wound healing.

3. A method for improving the innervation of regenerating tissue in a mammal, the method comprising:
identifying a mammal suffering from or at risk of suffering from a lack of innervation of regenerating tissue; and
administering an effective amount of two or more of the following substances: berry extract, chromium, zinc, trans-resveratrol, l-arginine, chlorophyll vitamin C, and aloe vera, wherein the administering improves the innervation of regenerating tissue.

4. A method for improving the healing of excisional dermal wounds in a mammal, the method comprising:
identifying a mammal suffering from or at risk of suffering from excisional dermal wounds; and
administering an effective amount of two or more of the following substances: berry extract, chromium, zinc, trans-resveratrol, l-arginine, chlorophyll, vitamin C, and aloe vera, wherein the administering improves healing of the excisional dermal wounds.

5. A method for improving peripheral circulation in a mammal, the method comprising:
identifying a mammal suffering from or at risk of suffering from poor peripheral circulation; and
administering an effective amount of two or more of the following substances: berry extract, chromium, zinc, trans-resveratrol, l-arginine, chlorophyll, vitamin C, and aloe vera, wherein the administering improves the peripheral circulation.

6. A method for preventing or healing neurodegeneration in a mammal, the method comprising:
identifying a mammal suffering from or at risk of suffering from neurodegeneration; and
administering an effective amount of two or more of the following substances: berry extract, chromium, zinc, trans-resveratrol, l-arginine, chlorophyll, vitamin C, and aloe vera, wherein the administering prevents or heals the neurodegeneration.

7. The method according to claim 5 wherein the administering is oral, and wherein the two or more substances are selected from the group consisting of berry extract, chromium, zinc, trans-resveratrol, l-arginine, chlorophyll and vitamin C.

8. The method according to claim 5 wherein the administering is topical, and wherein the two or more substances are selected from the group consisting of berry extract, chromium, zinc, trans-resveratrol, l-arginine, chlorophyll, vitamin C and aloe vera.

9. The method according to claim 5 wherein the two or more substances arc berry extract and chromium.

10. The method according to claim 5 wherein the mammal is suffering from diabetes mellitus.

11. A method for improving wound healing in a mammal, the method comprising identifying a mammal suffering from or at risk of suffering from wounds; and administering an effective amount of berry extract.

12. The method of claim 11 wherein the berry extract is from one or more berries selected from the group consisting of blueberry, bilberry, elderberry, cranberry, strawberry, raspberry, blackberry, dewberry, boysenberry, loganberry, youngberries, currant, gooseberry, juniper berry, huckleberry, thimbleberry, blackcap berry, mountain ash berry, salmonberry and wolfberry.

13. A composition for administration to reduce inflammation and enhance wound healing in a mammal, the composition comprising:
an amount of berry extract between:
a lower limit of approximately 3 mg human equivalency dosage (HED) per day; and
an upper limit of approximately 500 mg HED per day; an amount of chromium between:
a lower limit of approximately 10 μg HED per day; and
an upper limit of approximately 1000 μg HED per day; an amount of zinc between:
a lower limit of approximately 1.5 mg HED per day; and
an upper limit of approximately 75 mg HED per day;
an amount of trans-resveratrol between:
a lower limit of approximately 0.1 mg HED per day; and
an upper limit of approximately 50 mg HED per day;
an amount of l-arginine between:
a lower limit of approximately 50 mg HED per day; and
an upper limit of approximately 5000 mg HED per day;
an amount of chlorophyllin between:
a lower limit of approximately 10 mg HED per day; and
an upper limit of approximately 300 mg HED per day.
an amount of vitamin C between:
a lower limit of approximately 10 mg HED per day; and
an upper limit of approximately 10,000 mg HED per day.

14. The composition of claim 13 which is for topical application and further comprises:
an amount of aloe Barbendisis miller between:
a lower limit of approximately 0.01% (by weight); and
an upper limit of approximately 10% (by weight);

15. The method according to claim 5 wherein chromium is selected from the group consisting of chromium nicotinate, chromium polynicotinate, chromium picolinate, chromium chloride, chromium histidinate, and chromium present in yeast.

16. The method according to claim 5 wherein zinc is selected from the group consisting of zinc methionine, zinc sulfate, zinc polyascorbate. zinc oxide, zinc histidine, zinc gluconate, zinc citrate, zinc acetate, zinc picolinate, zinc alpha-ketoglutarate and zinc aspartate.

17. The method according to claim 5 wherein the berry extract further comprises one or more berries selected from the group consisting of blueberry, bilberry, elderberry, cranberry, strawberry, raspberry, blackberry, dewberry, boysenberry, loganberry, youngberries, currant, gooseberry, juniper berry, huckleberry, thimbleberry, blackcap berry, mountain ash berry, salmonberry and wolfberry.

18. The method according to claim 17 wherein the berry extract further comprises anthocyanins.

19. The method according to claim 5 wherein the trans-resveratrol is derived from Polygonum cuspidatum.

20. The method according to claim 19 wherein the trans-resveratrol is derived from alternate sources of trans-resveratrol plant or biological extracts, or biological or chemical synthesis.

21. The method according to claim 5 wherein chlorophyll comprises one or more components selected from the group consisting of water soluble chlorophyllin, sodium chlorophyllin, copper chlorophyllin, fat soluble chlorophyll A and fat soluble chlorophyll B.

22. The method according to claim 5 wherein vitamin C is selected from the group consisting of ascorbic acid, esterfied ascorbic acid and mineral salts of ascorbic acid including calcium ascorbate, magnesium ascorbate, potassium ascorbate and zinc ascorbate.

23. The method according to claim 5 wherein aloe vera further comprises aloe Brabendisis miller.
